(19) United States
(12) Patent Application Publication
(10) Pub. No.: US 2008/0051369 A1
(43) Pub. Date: Feb. 28, 2008

(54) Skin Cosmetic Material

(76) Inventors: Shu Uemura, Tokyo (JP); Chihiro Kaise, Tokyo (JP); Teruhisa Kaneko, Tokyo (JP); Hyung-Jun Kang, Gyeonggi-do (KR); Sang-woo Cho, Seoul (KR); Chang-Seo Park, Gyonggi-do (KR)

Correspondence Address: WENDEROTH, LIND & PONACK, L.L.P., 2033 K STREET N. W., SUITE 800, WASHINGTON, DC, 20006-1021, US

(21) Appl. No.: 11/791,612

(22) PCT Filed: Nov. 10, 2005
(86) PCT No.: PCT/JP05/20612

§ 371 Date: Aug. 6, 2007

(30) Foreign Application Priority Data
Nov. 25, 2004 (JP) 2004-339884

(51) Int. Cl. A61K 8/68; A61Q 1/00; A61Q 19/00

(52) U.S. Cl. 514/77

(57) ABSTRACT

[Object] To provide a cosmetic material having a superior moisture evaporation suppression ability and a high absorbed water volume.

[Solving Means] A cosmetic material contains sphingomyelin, and hence a high moisture evaporation suppression ability and a high absorbed water volume can be obtained.

### TECHNICAL FIELD

The present invention relates to a skin cosmetic material that has a superior moisture evaporation suppression ability and that increases an absorbed water volume, a skin cosmetic material composition, and a skin cosmetic treatment.

### BACKGROUND ART

Sphingomyelins broadly exist in organs of higher animals as well as in brain tissues, has a sphingoid base structure, and is one of sphingolipids having an amide bond in a hydrophilic portion in which an acid amide bond is formed with a fatty acid.

Heretofore, sphingomyelins have been used as an ingredient obtained from animals (particularly from cows) by extraction, has hardly been put on the market, and is one of lipids that have only been sold as a reagent. A highly pure product among sphingomyelins that have been available was only present in a very small amount, such as that of a reagent or the like. In addition, because of its strong intermolecular force, and in consideration of its price, it has been considerably difficult to add a highly pure product to a cosmetic material at a high concentration, and further, its features for skins have not also been clearly understood.

Among conventional cosmetic materials, some of them contain sphingomyelin as one phospholipid component; however, the above cosmetic materials use features of whole phospholipid containing lecithin and the like, and sphingomyelin is not used as an effective component of the cosmetic materials. In addition, by the reason described above, sphingomyelin has not been used as a compound for cosmetic materials so as to obtain a specific feature.

### DISCLOSURE OF INVENTION

### Problems to be Solved by the Invention

In recent years, egg- or milk-derived sphingomyelin was discovered, and the inventors of the present invention could clearly understood the properties of sphingomyelins by using the egg- or milk-derived sphingomyelin instead of a conventional cow-derived material. In particular, the finding was obtained that as one component of a cosmetic material, sphingomyelin is superior to other components of phospholipid, particularly, in terms of moisture evaporation suppression ability and absorbed water volume.

As an object of the present invention, a cosmetic material having the following features is provided by addition of egg- or milk-sphingomyelin. When the cosmetic material of the present invention is applied to skins, it exhibits a high moisture evaporation suppression ability, maintains an absorbed water volume at an optimal ratio, which is an index indicating a moisture volume retained in cornified layers, and has superior skin touch feeling. In addition, the present invention aims to solve the recent mad cow diseases by using egg- or milk-derived sphingomyelin instead of that derived from cows.

Furthermore, in order to continuously obtain a high moisture retention effect of skin cosmetic materials and makeup cosmetic materials, besides a barrier ability, it is also necessary to retain a moisture volume at a level that is essentially contained in skins. Accordingly, as still another object of the present invention, by designing sphingomyelin into a liposome form, a cosmetic material that more effectively exhibits the moisture retention effect is provided.

In addition, the present invention is to provide a skin cosmetic material composition that is used to add sphingomyelin to skin cosmetic materials as an effective component, and a skin cosmetic treatment for applying sphingomyelin to skins as an effective component.

Incidentally, as the sphingomyelin used for the applications described above, it is preferable that sphingomyelin, which is contained as a primary component of constituent components, be maintained at a high purity (15% or more).

### Means for Solving the Problems

In accordance with a first aspect of the present invention, there is provided a skin cosmetic material comprising sphingomyelin, wherein the skin cosmetic material has a high moisture evaporation suppression ability and has a high absorbed water volume.

According to a second aspect of the present invention, in the above skin cosmetic material, a liposome dispersing solution is included.

According to a third aspect of the present invention, in the above skin cosmetic material, the sphingomyelin is derived from either egg or milk.

According to a fourth aspect of the present invention, in the above skin cosmetic material, the sphingomyelin is a highly pure product.

According to a fifth aspect of the present invention, in the above skin cosmetic material, the sphingomyelin is contained at a concentration of 0.001 to 10.0 percent by weight.

According to a sixth aspect of the present invention, the above skin cosmetic material is a makeup cosmetic material.

According to a seventh aspect of the present invention, there is provided a skin cosmetic material composition comprising sphingomyelin, and the skin cosmetic material composition improves a moisture evaporation suppression ability and increases an absorbed water volume.

According to an eighth aspect of the present invention, in the above skin cosmetic material composition, the sphingomyelin is derived from either egg or milk.

According to a ninth aspect of the present invention, in the above skin cosmetic material composition, the sphingomyelin is a highly pure product.

According to a tenth aspect of the present invention, there is provided a skin cosmetic treatment comprising the step of applying a skin cosmetic material containing sphingomyelin, whereby a moisture evaporation suppression ability is improved, and an absorbed water volume is increased.

According to an eleventh aspect of the present invention, in the above skin cosmetic treatment, the sphingomyelin is derived from either egg or milk.

According to a twelfth aspect of the present invention, in the above skin cosmetic treatment, the sphingomyelin is a highly pure product.

Advantages

According to the present invention, a skin cosmetic material is provided that contains sphingomyelin and that has a high moisture evaporation suppression ability and a high absorbed water volume. According to this type of skin cosmetic material, compared to conventional cosmetic materials using phospholipids such as lecithin, a cosmetic material can be provided that exhibits specific features of sphingomyelin and that has superior moisture evaporation suppression ability and absorbed water volume.

In addition, according to the present invention, in the skin cosmetic material described above, since the liposome dispersing solution is included, the sphingomyelin can be made into a liposome form; hence, an essential moisture volume contained in skins can be retained, and a high moisture retention effect can be continuously obtained.

In addition, according to the present invention, in the skin cosmetic material described above, since the sphingomyelin is derived from either egg or milk, the sphingomyelin can be effectively added to a cosmetic material at a high concentration, and the barrier ability and moisture retention ability can be enhanced; hence, a highly functional moisture retaining cosmetic material can be provided that enable skins to have a high moisture retention ability. In addition, although it has been difficult by a conventional reagent derived from cows, when sphingomyelin derived from either egg or milk is used, specific features of sphingomyelin in cosmetic materials can be clearly understood. Furthermore, since the sphingomyelin derived from either egg or milk is used, problems of the recent mad cow diseases caused by an ingredient derived from cows can be solved.

In addition, according to the present invention, in the above skin cosmetic material, since the sphingomyelin is a highly pure product, a highly pure sphingomyelin can be contained at a high concentration.

In addition, according to the present invention, in the above skin cosmetic material, since the sphingomyelin is contained at a concentration of 0.001 to 10.0 percent by weight, the sphingomyelin can be contained as an effective component in a cosmetic material, and hence the features of the sphingomyelin for skins can be effectively obtained.

In addition, according to the present invention, since the above skin cosmetic material is a makeup cosmetic material, the moisture evaporation suppression ability and the function of the absorbed water volume, which are caused by the sphingomyelin and which are suitable for applications of makeup cosmetic materials, can be effectively obtained.

In addition, according to the present invention, a cosmetic material composition can be provided that contains sphingomyelin, and that improves a moisture evaporation suppression ability and increases an absorbed water volume. According to the cosmetic material composition as described above, sphingomyelin can be contained as an effective component in a skin cosmetic material, and hence the moisture evaporation suppression ability and the absorbed water volume can be enhanced.

In addition, according to the present invention, a skin cosmetic treatment can be provided that improves a moisture evaporation suppression ability and increases an absorbed water volume for skins by applying thereto a skin cosmetic material containing sphingomyelin. By the skin cosmetic treatment as described above, sphingomyelin can be applied to skins as an effective component of a skin cosmetic material, and hence the moisture evaporation suppression ability and the absorbed water volume for skins can be enhanced.

### BEST MODE FOR CARRYING OUT THE INVENTION

Hereinafter, embodiments of the present invention will be described. However, the present invention is not limited to the embodiments to be described by way of example.

A skin cosmetic material according to the present invention contains sphingomyelin, has a high moisture evaporation suppression ability, and imparts a high absorbed water volume to skins. A skin cosmetic material of one embodiment according to the present invention is prepared by compounding egg- or milk-derived sphingomyelin with components generally used for skin cosmetic materials.

The sphingomyelin used in this embodiment is an egg- or milk-derived product, is not a reagent grade, and is prepared as an ingredient for cosmetics. When this sphingomyelin is contained in a cosmetic material formulation, compared to a liposome single cosmetic material formed using lecithin, which is generally believed to have a high skin affinity, a barrier ability, and a moisture retention ability for skins, a cosmetic material formulation having a high barrier ability and a high moisture retention ability (absorbed water volume) can be prepared by the presence of amide bonds in molecules. In particular, because of the high barrier ability of suppressing moisture evaporation in confined layers, a highly functional moisture retaining cosmetic material having a superior moisture retention ability can be prepared, and as a result, besides the adjustment of wrinkles, which are caused by dry skins, and skin turnover, an antiinflammatory effect can also be expected.

When the sphingomyelin described above is used, a skin cosmetic material composition can be manufactured which contains sphingomyelin and which improves the moisture evaporation suppression ability and increases the absorbed water volume.

In the cosmetic material according to this embodiment, the egg- or milk-derived sphingomyelin is preferably in the form of powder or solid, and the content thereof is preferably in the range of 0.001 to 10.0 percent by weight. When the content is less than 0.001 percent by weight, a high barrier ability and a high absorbed water volume, which are specific features of sphingomyelin, cannot be effectively obtained, and it becomes difficult to obtain a high moisture retention effect. In addition, when the content exceeds 10.0 percent by weight, it is not preferable since degradation in stability, such as separation, discoloration, and/or change in smell, or adverse influence on functionality of the skin cosmetic material may occur in some cases. In addition, the purity of the sphingomyelin used in the present invention, which is a primary component of constituent components, is preferably set to 15% or more.

The skin cosmetic materials are not particularly limited and, for example, may be formed into cosmetics, such as an emulsion, cream, skin lotion, face pack, face wash, massage-relating agent, and makeup cosmetic materials including liquid foundation and powder foundation; body cosmetic materials; and quasi-drugs.

In addition, as the makeup cosmetic materials, besides the above liquid foundation and powder foundation, for example, there may be mentioned a lipstick, lip cream, cheek powder, eye shadow, face powder, and concealer.

Besides the above essential components, components added to general cosmetic materials, such as an oil compound, powder, surfactant, purified water, lower alcohol, high molecular weight compound, gelling agent, ultraviolet absorber, ultraviolet scattering agent, oxidation inhibitor, dye, antiseptic, fragrance, essence component may be optionally used as long as it may not have any adverse influence on the effects of the present invention.

As a base material forming a film of liposome, for example, there may be mentioned natural or synthetic phospholipids, such as phosphatidylcholine, phosphatidylethanolamine, phosphatidylinositol, phosphatidylserine, phosphatidylglycerol, phosphatidic acid, lysophosphatidylcholine, sphingomyelin, egg-yolk lecithin, soybean lecithin, and their hydrogen-added phospholipids; cholesterols, such as cholesterol and its alkyester; phytosterol and its alkylester; glycoglycerolipid; acylglucoside such as cetylgalactoside; dialkyl type synthetic surfactants; N-acyl-sphingosine or its sulfuric acid ester; N-acyl-glycosphingolipid; and N-higher acyl glutathione. Those mentioned above may be used alone or in combination. In addition, in order to stabilize the liposome or to improve the phase transition temperature, for example, a polyalcohol, a higher alcohol, or a higher fatty acid may be added whenever necessary.

The composition standard is in the range of 50 to 99 percent by weight and is preferably in the range of 50 to 90 percent by weight.

As fats and fatty oils, for example, jojoba oil, castor oil, olive oil, soybean oil, coconut oil, palm oil, cacao oil, mink oil, and turtle oil may be mentioned.

As hydrocarbons, for example, there may be mentioned liquid paraffin, vaseline, microcrystalline wax, and squalane.

As waxes, for example, there may be mentioned beeswax, lanolin, carnauba wax, and candelilla wax.

As fatty acids, for example, there may be mentioned myristic acid, palmitic acid, stearic acid, oleic acid, isostearic acid, and lauric acid.

As synthetic esters, for example, there may be mentioned isopropyl myristate, isopropyl palmitate, butyl oleate, myristyl myristate, octyldecyl myristate, propylene glycol monostearate, myristyl lactate, isostearyl malate, glycerin monostearate, and distearyldimethyl ammonium chloride.

In general, the fats and fatty oils, hydrocarbons, waxes, fatty acids, and synthetic esters are contained at a total concentration of 0 to 30 percent by weight.

As alcohols, for example, there may be mentioned ethanol, 1,3-butylene glycol, propylene glycol, lauryl alcohol, cetanol, stearyl alcohol, and oleyl alcohol.

In general, the alcohols are contained at a concentration of 0 to 25 percent by weight.

As surfactants, for example, there may be mentioned glycerin fatty acid esters, sorbitan fatty acid esters, polyoxyethylene fatty acid esters, polyethylene sorbitan fatty acid esters, polyoxyethylene alkyl ethers, coconut oil fatty acid monoethanolamide, polyoxyethylene hardened castor oil, sodium lauryl sulfate, polyoxyethylene glyceryl pyroglutamate isostearate, sodium alkylbenzene sulfonate, polyoxyethylene stearyl ether, dialkyl sulfosuccinate, cetyl pyridinium bromide, n-octadecyl trimethylammonium chloride, monoalkyl phosphate, N-acylglutamic acid, N-acylglutamine, sucrose fatty acid ester, polyoxyethylene sorbitan monostearate, polyoxyethylene lauryl ether sodium sulfate, and polyoxyethylene-reduced lanolin.

The surfactant is contained generally at a concentration of 0 to 10 percent by weight.

As thickeners, for example, there may be mentioned carboxyvinyl polymer, methylpolysiloxane, dextran, carboxymethyl cellulose, carrageenin, and hydroxypropylmethyl cellulose.

The thickener is contained generally at a concentration of 0 to 5 percent by weight.

As moisturizers, for example, there may be mentioned glycerin, propylene glycol, 1,3-butylene glycol, pyroglutamic acid, acetylglutamate, hyaluronic acid, procyanidin, and L-arginine.

The moisturizer is contained generally at a concentration of 0 to 25 percent by weight.

As antiseptics, for example, there may be mentioned benzoic acid, salicylic acid, dehydroacetic acid or its salt, phenols such as p-oxybenzoate, triclosan and halocarbane.

The antiseptic is contained generally at a concentration of 0 to 0.3 percent by weight.

As fragrances, any type of fragrant may be used as long as it is generally used for cosmetic materials.

As pigments, for example, there may be mentioned iron oxide, titanium dioxide, zinc oxide, kaolin, and talc.

The pigment is contained generally at a concentration of 0 to 5 percent by weight.

As chemicals, for example, there may be mentioned wheat germ oil, vitamin A, vitamin B2, vitamin E, ascorbic acid-2-phosphate magnesium or sodium, D-pantothenol alcohol, dipotassium glycyrrhizinate, glutathione, UV absorbers, chelating agents, plant extracts, and microbial metabolites/extracts.

The chemical is contained generally at a concentration of 0 to 5 percent by weight.

As water, for example, there may be mentioned tap water, mineral water, brine water, sea water, deep sea water, ultrapure water, polar ice-derived water, mineral-containing water, and purified water.

The water may be contained at an optional concentration.

In addition, since surface sea water falls and forms a layer of deep sea water, the deep sea water according to the present invention is specific low-temperature and clean water containing plenty of nutrient salts. The water-intake sea area is 1,000 to 4,000 m and is preferably 2,000 to 3,000 m, and the water-intake depth is 250 to 500 m and is preferably 300 to 400 m.

Since containing plenty of minerals, the deep sea water has superior moisture retention properties and also has superior penetrating feeling; hence, it is believed that penetration of effective components contained in cosmetic materials into skins can be facilitated.

The form of cosmetic material may be optionally designed, and for example, either a soluble emulsion form or a dispersion form may be used.

As cosmetic material products that use the cosmetic material according to this embodiment, there may be mentioned, for example, skin care products such as a skin lotion, emulsion, essence, cream, face wash, pack, and gel; and products including an emulsion foundation.

In addition, only by applying the cosmetic material according to this embodiment to skins, a skin cosmetic treatment can be provided that improves a moisture evaporation suppression ability and an absorbed water volume for skins.

### EXAMPLES

Hereinafter, the examples of the present invention will be described in detail; however, the present invention is not limited thereto.

### Example 1

(Test for Evaluating Effect of Moisture Evaporation Suppression Ability of Sphingomyelin)

An aqueous solution containing milk-derived sphingomyelin as Example 1, an aqueous liposome solution containing corn sphingoglycolipid of plant-derived sphingoglycolipid as Comparative Example 1, an aqueous liposome solution using phospholipid (soybean lecithin) as Comparative Example 2, and an aqueous liposome solution using phospholipid (egg-yolk lecithin) as Comparative Example 3 were prepared, and the moisture evaporation suppression ability (barrier ability) of each ingredient was examined.

### Test Example 1

After an aqueous solution containing each ingredient at a concentration of 1 percent by weight was prepared by a known method, that is, by the Bangham method, a predetermined amount of this solution was applied to an artificial model skin, followed by sufficient drying at room temperature. Subsequently, after this artificial skin was placed at an upper part of a cup containing water and was held for 24 hours at 37° C. and at a humidity of 40%, the moisture evaporation rate in the cup was measured. The moisture evaporation rate thus measured was regarded as the moisture evaporation suppression ability. The results are shown in Table 1.

TABLE 1

| | | Moisture Evaporation |
|---|---|---|
| | | Suppression Ability (%) |
| Example 1 | Milk-derived sphingomyelin | 51 |
| Comparative | Corn sphingoglycolipid | 32 |
| Example 1 | | |
| Comparative | Phospholipid (soybean | 25 |
| Example 2 | lecithin) | |
| Comparative | Phospholipid (egg-yolk | 20 |
| Example 3 | lecithin) | |

According to the above results, it was found that compared to the phospholipids (Comparative Examples 2 and 3), which were generally believed to have a high moisture evaporation suppression ability, and compared to the corn sphingoglycolipid (Comparative Example 1), the milk-derived sphingomyelin of Example 1 has a considerably high moisture evaporation suppression ability. In addition, in Example 1, it was found that when the milk-derived sphingomyelin was contained, an aqueous liposome solution can be obtained that has a lipid film capable of suppressing the moisture evaporation to 50% or more.

### Example 2

(Test for Evaluating Effect of Absorbed Water of Sphingomyelin)

An aqueous solution containing milk-derived sphingomyelin as Example 2, an aqueous solution containing rice-derived sphingoglycolipid as Comparative Example 4, an aqueous solution of phospholipid (soybean lecithin) as Comparative Example 5, and an aqueous solution of phospholipid (egg-yolk lecithin) as Comparative Example 6 were prepared, and the absorbed water volume of each ingredient was examined.

### Test Example 2

After preparation was performed by adding each ingredient to purified water so that the concentrations thereof were different from each other, annealing was then performed; as a result, uniformly dispersed aqueous solutions were formed. By using the solutions thus formed, in accordance with a known method, that is, with DSC (differential scanning calorie analysis), the absorbed water of each solution was measured. The results are shown in Table 2.

TABLE 2

| | | Absorbed water |
|---|---|---|
| | | volume (%) |
| Example 2 | Milk-derived sphingomyelin | 19 |
| Comparative | Rice-derived | 9 |
| Example 4 | sphingoglycolipid | |
| Comparative | Phospholipid (soybean | 15 |
| Example 5 | lecithin) | |
| Comparative | Phospholipid (egg-yolk | 13 |
| Example 6 | lecithin) | |

According to the above results, it was found that the milk-derived sphingomyelin of Example 2 has a high absorbed water volume as compared to that of the phospholipids (Comparative Examples 5 and 6) which are generally believed to have a high absorbed water volume, and furthermore, compared to that of the rice-derived sphingoglycolipid (Comparative Example 4) which is expected to have a high absorbed water volume since sugar was contained in the structure thereof, the milk-derived sphingomyelin of this example has a considerably high absorbed water volume.

### Example 3

(Test for Evaluating Effect of Barrier Ability for Skins by Cream)

After a predetermined amount of a cream containing milk-derived sphingomyelin was applied to an artificial model skin, this model skin was used in order to examine how much the moisture evaporation could be prevented when the sphingomyelin was actually contained in cosmetics.

(Formulation Example 1)
(On percent by weight basis)

| | |
|---|---|
| Deep sea water (processed by | 50.0 |
| reverse osmosis filtration) | |
| Jojoba oil | 10.0 |
| Phospholipid | 1.5 |
| Cholesterol | 0.7 |
| Glycerin fatty acid ester | 2.0 |
| Higher alcohol | 3.0 |
| Polyalcohol fatty acid ester | 0.5 |
| Carboxyvinyl polymer | 0.05 |
| Glycerin | 7.0 |
| 1,3-butylene glycol | 7.0 |
| L-arginine | 0.1 |
| Moisturizer | 0.5 |
| Antiseptic | 0.5 |
| Purified water | adjusted to make |
| | the total 100 |

Three types of creams were prepared by adding 1 percent by weight of sphingomyelin in Example 3, 1 percent by weight of corn sphingoglycolipid in Comparative Example 7, and 1 percent by weight of phospholipid (soybean lecithin) in Comparative Example 8 to the above standard formulation, and in addition, as Comparative Example 9, a cream was also prepared that contained no lipid. The manufacturing method was performed in accordance with a known method for preparation, so that the creams were obtained. The deep sea water (processed by reverse osmosis filtration) used in this example was water obtained by processing deep sea raw water by reverse osmosis filtration.

### Test Example 3

After a predetermined amount of each cosmetic material bulk prepared in accordance with a known method was applied to an artificial model skin, sufficient drying was performed at room temperature. Subsequently, after this artificial skin was placed at an upper part of a cup containing water and was held for 24 hours at 37° C. and at a humidity of 40%, the moisture evaporation rate in the cup was measured. The moisture evaporation rate thus measured was regarded as the moisture evaporation suppression ability. The results are shown in Table 3.

TABLE 3

| | | Moisture |
|---|---|---|
| | | evaporation |
| | | suppression |
| | | ability (%) |
| Example 3 | Cream containing 1 percent by weight of | 60 |
| | milk-derived sphingomyelin | |
| Comparative | Cream containing 1 percent by weight of | 30 |
| Example 7 | corn sphingoglycolipid | |
| Comparative | Cream containing 1 percent by weight of | 18 |
| Example 8 | phospholipid (soybean lecithin) | |
| Comparative | Cream containing no lipid | 9 |
| Example 9 | | |

According to the above results, it was found that compared to the cream containing corn sphingoglycolipid (Comparative Example 7), that containing phospholipid (Comparative Example 8), and that containing no lipid, the cream containing 1 percent by weight of milk-derived sphingomyelin of Example 3 has a considerably high moisture evaporation suppression ability.

As described above, also in the case in which sphingomyelin is actually contained in a cream, compared to conventional phospholipid and sphingoglycolipid, a high moisture evaporation suppression ability can be obtained; hence, as a result, by using sphingomyelin as a cosmetic material, a highly functional moisture retaining cream can be realized.

1. A skin cosmetic material comprising: sphingomyelin as an effective component, wherein the skin cosmetic material has a high moisture evaporation suppression ability and has a high absorbed water volume.

2. The skin cosmetic material according to claim 1, wherein a liposome dispersing solution is included.

3. The skin cosmetic material according to claim 1, wherein the sphingomyelin is derived from either egg or milk.

4. The skin cosmetic material according to claim 1, wherein the sphingomyelin is a highly pure product.

5. The skin cosmetic material according to claim 1, wherein the sphingomyelin is contained at a concentration of 0.001 to 10.0 percent by weight.

6. The skin cosmetic material according to claim 1, wherein the skin cosmetic material is a makeup cosmetic material.

7. A moisture evaporation suppressor comprising: sphingomyelin as an effective component.

8. The moisture evaporation suppressor according to claim 7, wherein the sphingomyelin is derived from either egg or milk.

9. The moisture evaporation suppressor according to claim 7, wherein the sphingomyelin is a highly pure product.

10. A skin cosmetic treatment comprising the step of applying the skin cosmetic material according to claim 1 to skin, whereby the moisture evaporation suppression ability is improved, and the absorbed water volume is increased.

11. The skin cosmetic treatment according to claim 10, wherein the sphingomyelin is derived from either egg or milk.

12. The skin cosmetic treatment according to claim 10, wherein the sphingomyelin is a highly pure product.

13. An absorbed water-volume increaser comprising: sphingomyelin as an effective component.

14. The absorbed water-volume increaser according to claim 13, wherein the sphingomyelin is derived from either egg or milk.

15. The absorbed water-volume increaser according to claim 13, wherein the sphingomyelin is a highly pure product.
